(19) United States
(12) Patent Application Publication
(10) Pub. No.: US 2025/0250440 A1
(43) Pub. Date: Aug. 7, 2025

(54) Triarylmethane Dyes

(71) Applicant: Kinnos Inc., Brooklyn, NY (US)

(72) Inventors: Pau OLIVERES, Brooklyn, NY (US); Matthew JUROW, Brooklyn, NY (US); Dorothy JONES, Brooklyn, NY (US); Kara NG, Brooklyn, NY (US)

(21) Appl. No.: 19/018,038

(22) Filed: Jan. 13, 2025

Related U.S. Application Data
(63) Continuation of application No. PCT/US23/28556, filed on Jul. 25, 2023.
(60) Provisional application No. 63/391,864, filed on Jul. 25, 2022.

(51) Int. Cl. C09B 11/24; C09B 69/00

(52) U.S. Cl. CPC C09B 11/24; C09B 69/00

(57) ABSTRACT

The present invention provides modified triarylmethane dyes comprising the structure shown in Formula 3,

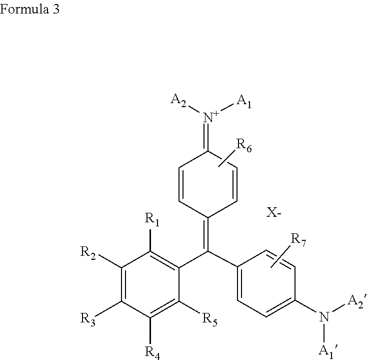

wherein R₁, R₂, R₃, R₄, R₅, R₆, R₇, A₁, A₂, A_{1′}, and A_{2′} are defined herein.

### RELATED APPLICATIONS

This application is a continuation of International Application No. PCT/US23/28556, which designated the United States and was filed on Jul. 25, 2023, published in English, which claims the benefit of U.S. Provisional Application No. 63/391,864, filed on Jul. 25, 2022. The entire teachings of the above applications are incorporated herein by reference.

### FIELD OF THE INVENTION

The present invention relates to triarylmethane dyes and to the use of the triarylmethane dyes.

### BACKGROUND OF THE INVENTION

The family of triarylmethane dyes produce brilliant colors, tunable across broad sections of the visible spectrum. Triarylmethane (TAM) dyes are used widely in producing paper dyes, printing inks, ballpoint pen pastes, food, cosmetics, and a growing range of consumer products. Most known triarylmethane dyes are developed from the central core with the purpose of increasing resistance to decolorization. It is an object of the present invention to provide triarylmethane dyes wherein the extent of the aromatic system and the concomitant optical behavior are controlled.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows a mass spectrum that corroborates the mass to charge ratio of one embodiment of an acid triarylmethane (TAM) dye molecule according to the invention.

FIG. 2 shows the ¹H NMR spectra of the acid TAM dye and confirms the atomic structure of the dye.

FIG. 3 further confirms the atomic structure of the acid TAM dye compound via ¹H NMR by assigning the proton shifts observed in the spectra to the molecule.

FIG. 4 shows the mass spectrum that corroborates the mass to charge ratio of one embodiment of a PEG TAM dye molecule according to the invention.

FIG. 5 shows the ¹H NMR spectra of PEG TAM dye compound and confirms the atomic structure.

FIG. 6 further confirms the atomic structure of the PEG TAM dye compound via ¹H NMR by assigning the proton shifts observed in the spectra to the molecule. In the structure shown, one PEG chain shows the proton labeling; the same labeling can be applied to all four PEG chains.

FIG. 7 depicts the UV spectrum of equimolar (25 μM) solutions of Acid Green 9, meta chloro acid triarylmethane, and meta chloro PEG triarylmethane demonstrating a pronounced change in relative absorbance and peak positioning, tabulated in Table 6. The meta chloro acid triarylmethane presents a greener hue than Acid Green 9 in aqueous solution.

FIG. 8 depicts the normalized absorbance at λₘₐₓ vs time graph of 25 μM dye solutions in phosphate buffer (pH=8, 0.1 M). The plot demonstrates the difference in alkali fastness between Acid Green 9, meta chloro acid triarylmethane, and meta chloro PEG triarylmethane.

FIG. 9 depicts the normalized absorbance at λₘₐₓ vs time graph of 1:1 25 μM unbuffered dye solution and 0.5% H₂O₂. The graph shows how meta chloro acid triarylmethane is significantly less oxygen fast than its closest related analogue, and meta chloro PEG triarylmethane is even less oxygen fast. Hydrogen peroxide on its own is not a powerful enough oxidant to fade most triarylmethane dyes. Acid Green 9 is susceptible to fast fading by hydrogen peroxide at high pHs only. FIG. 9 shows how, even in a system that is not buffered at an alkaline pH, both meta chloro acid triarylmethane and meta chloro PEG triarylmethane will still fade in the presence of hydrogen peroxide.

### DESCRIPTION OF THE INVENTION

Triarylmethanes are known to practitioners of the art as a class of molecules generally represented by the Formula 1, where any combination of the R groups are aryl groups that may have additional functionalization including but not limited to amino (primary, secondary, or tertiary), hydroxy, halogens (bromo, chloro, fluoro, iodo), nitro, methoxy, carboxy, alkyl, aryl, alkoxy, and sulfonic acid. The counter ion, X can be provided from an external molecule or an inner sphere charged group related to any of the R groups.

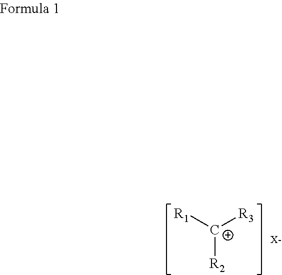

Due to the resonance that exists within the molecule, the structure of the triarylmethane dye can be depicted in different ways, as shown in Formula 2, wherein structures 1, 1′ and 1″ are all equivalent. Based on convention, structure 1 will be used herein.

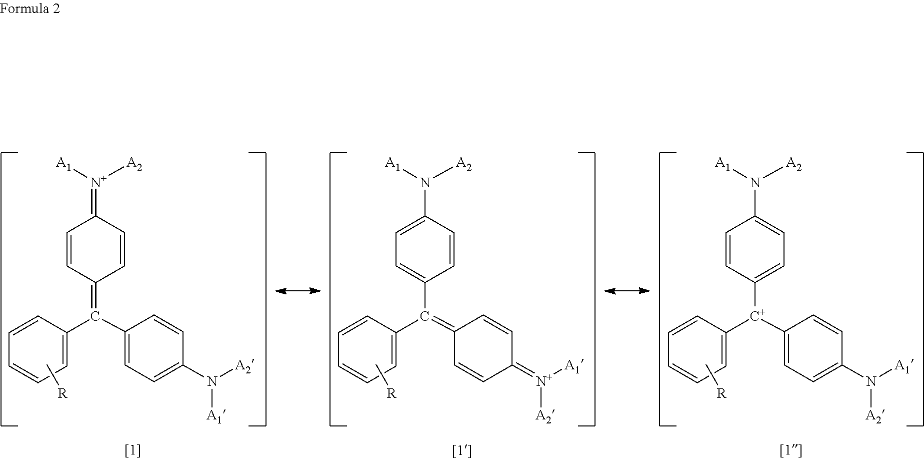

The present invention provides modified triarylmethane dyes comprising the structure shown in Formula 3

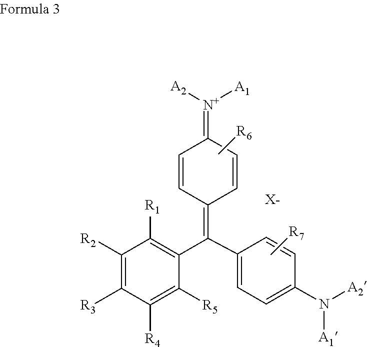

wherein
R₁, R₂, R₃, R₄, R₅, R₆ and R₇ are independently selected from hydrogen, halogen, C₁-C₁₆ alkyl, amino, nitro, methoxy, carboxy, aryl, alkoxy, and sulfonic acid;
A₁, A₂, A_{1′}, and A_{2′} are independently selected from hydrogen, alkyl, aryl, alkaryl, or alkoxy, provided that at least one of A₁, A₂, A_{1′}, and A_{2′} is alkyl, aryl, alkaryl, or alkoxy.

In embodiments, R₁, R₂, R₃, R₄, R₅, R₆ and R₇ are independently selected from hydrogen, halogen, C₁-C₁₆ alkyl, amino, nitro, methoxy, carboxy, aryl, alkoxy, and sulfonic acid, provided that at least one of R₁, R₂, or R₃ are halogen.

In one embodiment, modified triarylmethane dyes comprising the structure shown in Formula 4,

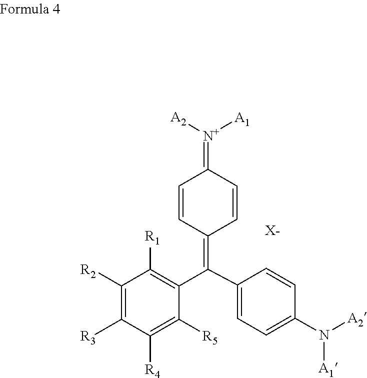

wherein
R₁, R₂, R₃, R₄, and R₅ are independently selected from hydrogen, halogen, C₁-C₁₆ alkyl, amino, nitro, methoxy, carboxy, aryl, alkoxy, and sulfonic acid, provided that at least one of R₁, R₂, or R₃ are halogen;
A₁, A₂, A_{1′}, and A_{2′} are independently selected from hydrogen, alkyl, aryl, alkaryl, or alkoxy, provided that at least one of A₁, A₂, A₁, and A₂′ is alkyl, aryl, alkaryl, or alkoxy.

In embodiments, R₁, R₂, R₃, R₄, and R₅ are independently selected from hydrogen, halogen, C₁-C₁₆ alkyl, amino, nitro, methoxy, carboxy, aryl, alkoxy, and sulfonic acid, provided that at least one of R₁, R₂, or R₃ are halogen.

In any embodiment herein, halogen is selected from fluorine, bromine, chlorine, or iodine. In any embodiment herein, the halogen is fluorine. In any embodiment herein, the halogen is bromine. In any embodiment herein, the halogen is chlorine. In any embodiment herein, the halogen is iodine.

In any embodiment herein, the amino is a primary, secondary, or tertiary amino. In any embodiment herein, the amino is a primary amino. In any embodiment herein, the amino is a secondary amino. In any embodiment herein, the amino is a tertiary amino.

In embodiments, only one of R₁, R₂, or R₃, is halogen and the other two are hydrogen. In embodiments, when R₁ is halogen, R₂ and R₃ are hydrogen and R₁ is selected from fluorine, bromine, chlorine, or iodine. In embodiments, when R₂ is halogen, R₁ and R₃ are hydrogen and R₂ is selected from fluorine, chlorine, bromine, or iodine. In embodiments, when R₃ is halogen, R₁ and R₂ are hydrogen and R₃ is selected from fluorine, bromine, or iodine.

In embodiments, R₄, and R₅ are hydrogen.

In embodiments, R₆, and R₇ are hydrogen.

In one embodiment, modified triarylmethane dyes comprising the structure shown in Formula 4,

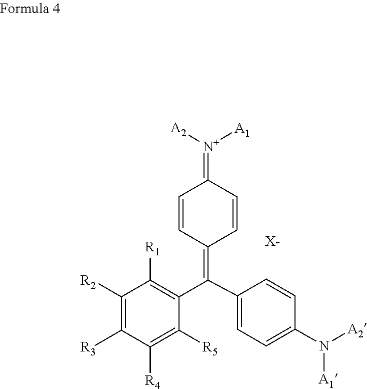

wherein
R₁, R₂, R₃, are independently selected from hydrogen or halogen, provided only one of R₁, R₂, or R₃, is halogen, and provided that,
when R₁ is halogen, R₂ and R₃ are hydrogen and R₁ is selected from fluorine, bromine, or iodine,
when R₂ is halogen, R₁ and R₃ are hydrogen and R₂ is selected from fluorine, chlorine, bromine, or iodine, and
when R₃ is halogen, R₁ and R₂ are hydrogen and R₃ is selected from fluorine, bromine, or iodine;
   R₄, and R₅ are hydrogen; and
A₁, A₂, A_{1′}, and A_{2′} are independently selected from hydrogen, alkyl, aryl, alkaryl, or alkoxy, provided that at least one of A₁, A₂, A₁, and A₂′ is alkyl, aryl, alkaryl, or alkoxy.

In embodiments, A₁, A₂, A₁′, and A₂′ are independently selected from hydrogen, alkyl, aryl, alkaryl or alkoxy, provided that at least one of A₁, A₂, A_{1′}, and A_{2′} is alkyl, aryl, alkaryl or alkoxy.

In embodiments, when R₁ is halogen, A₁, A₂, A₁′, and A₂′ are independently selected from hydrogen, alkyl, aryl, alkaryl, or alkoxy.

As used in any embodiment herein, the term halogen refers to any element in the group containing fluorine, chlorine, bromine, and iodine.

As used in any embodiment herein, the term “alkyl” describes both substituted or unsubstituted straight and branched carbon chains. Preferred alkyl groups are those containing from one to fifteen carbon atoms (i.e., C₁-C₁₅ alkyl), more preferably C₁-C₁₀ alkyl, more preferably C₁-C₅ alkyl. In embodiments, the alkyl is selected from methyl, ethyl, propyl, 1-methylethyl, butyl, 1-methylpropyl, 2-methylpropyl, pentyl, 1-methylbutyl, 2-methylbutyl, 3-methylbutyl, 1,1-dimethylpropyl, 1,2-dimethylpropyl, 2,2-dimethylpropyl, each of which can be optionally substituted. Preferably, the alkyl is substituted or unsubstituted ethyl. Preferably, the alkyl is unsubstituted ethyl.

As used in any embodiment herein, the term “aryl” or “aromatic group” are used interchangeably to describe either substituted or unsubstituted single-ring or polycyclic ring system. Polycyclic rings may have two or more rings in which two carbons are common to two adjoining rings (“fused” rings) wherein at least one of the rings is aromatic, e.g., the other rings can be cycloalkyls, cycloalkenyls, aryl, heterocycles, and/or heteroaryls. Preferred aryl groups are those containing six to thirty carbon atoms (i.e., C₆-C₃₀ aryl), preferably six to twenty carbon atoms (i.e., C₆-C₂₀ aryl), more preferably, six to twelve carbon atoms (i.e., C₆-C₁₂ aryl). In embodiments, the aryl comprises six carbon atoms, ten carbons, or twelve carbons. Suitable aryl groups include, but are not limited to, phenyl, biphenyl, triphenyl, triphenylene, tetraphenylene, naphthalene, anthracene, phenalene, phenanthrene, fluorene, pyrene, chrysene, perylene, and azulene, preferably phenyl, biphenyl, triphenyl, benzyl sulfonic acid, triphenylene, fluorene, and naphthalene, each of which can be optionally substituted.

As used in any embodiment herein, the term “aralkyl” describes an alkyl group comprised of 1 to 15 carbon atoms that has as a substituent an aromatic group. Additionally, the aralkyl group may be optionally substituted.

As used in any embodiment herein, the term “alkoxy” describes any alkyl group bonded to an oxygen group. In embodiments, the alkoxy group includes but is not limited to an ether, ester, amide, carboxylic acid, or alcohol. Also included in this definition are structures featuring periodic insertions of an oxygen atom into a carbon chain of less than 20 carbons atoms and terminated with an alcohol or an alkyl group such as, but not limited to, polyethylene glycol. Preferred alkoxy groups are polyethylene glycol chains, preferably with 1-10 ethylene glycol repeat units, more preferably 2-8 ethylene glycol repeat units. In embodiments, the polyethylene glycol chains contain 5 ethylene glycol repeat units.

In any embodiment herein, A₁, A₂, A_{1′}, and A_{2′} provide a solubilizing group to the structure of formula I. As used herein, the term “solubilizing group” describes a terminal carboxylic acid, sulfonate, benzyl sulfonic acid, ester, or alcohol group introduced to improve solubility in polar solvents (water, alcohol).

Selective halogenation of the dye molecule on the R₁-R₃ positions alters the electronic structure, making the pi system more susceptible to interruption or cleavage that results in loss of color, effectively modulating the rate of decolorization.

In embodiments, the amine groups can be substituted to create molecules such that:
i) A₁ and A₂ can be the same or can be different;
ii) A₁ and A₂′ can be the same or can be different;
iii) A₁ and A₁′ can be the same or can be different;
iv) A₂ and A₁′ can be the same or can be different;
v) A₂ and A₂′ can be the same or can be different; or
vi) A₁′ and A₂′ can be the same or can be different.

As used herein, the term “optionally substituted” means that the group in question can be substituted at one or more positions by any one or combination of members of the following list: halogen, hydrogen, alkyl, aryl, alkoxy, aralkyl, solubilizing groups.

In any embodiment herein, A₁ and A₁′ are the same and A₂ and A₂′ are the same. In embodiments, A₁ and A₁′ are an alkyl and A₂ and A₂′ are an aryl. In embodiments, A₁ and A₁′ are a C₁-C₅ alkyl and A₂ and A₂′ are a C₆-C₁₂ aryl. In embodiments, A₁=A₁′=ethyl and A₂=A₂′=benzyl sulfonic acid.

In any embodiment herein, A₁, A₁′, A₂ and A₂′ are alkoxy. In embodiments, A₁, A₁′, A₂ and A₂′ are polyethylene glycol (PEG). Preferably A₁, A₁′, A₂ and A₂′ are independently polyethylene glycol with 1-10 ethylene glycol repeat units. Preferably A₁, A₁′, A₂ and A₂′ are independently polyethylene glycol with 2-8 ethylene glycol repeat units. In embodiments, A₁, A₁′, A₂ and A₂′ are polyethylene glycol chains containing 5 ethylene glycol repeat units. In embodiments, A₁, A₁′, A₂ and A₂′ are all the same.

Non-limiting examples of compounds according to the invention are shown in Tables 1-4.

TABLE 1

| | |
|---|---|
| 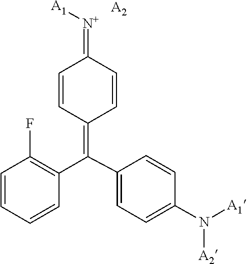 | [2] |
| 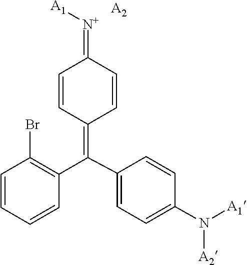 | [3] |
| 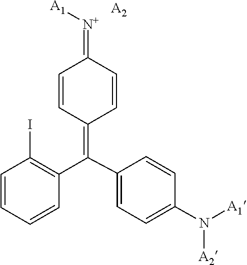 | [4] |
| 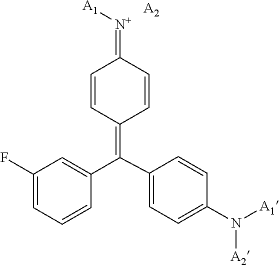 | [5] |
| 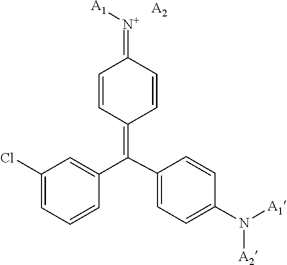 | [6] |
| 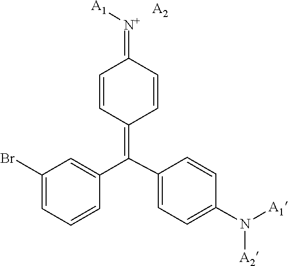 | [7] |
| 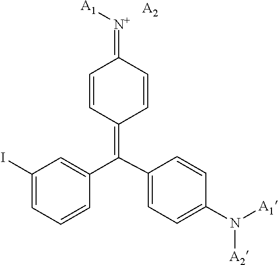 | [8] |
| 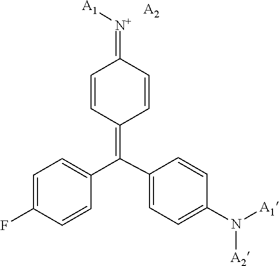 | [9] |
| 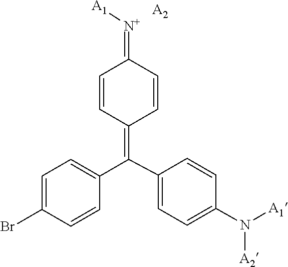 | [10] |
| 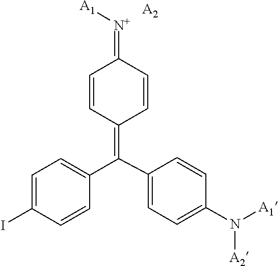 | [11] |

TABLE 2

| | |
|---|---|
| 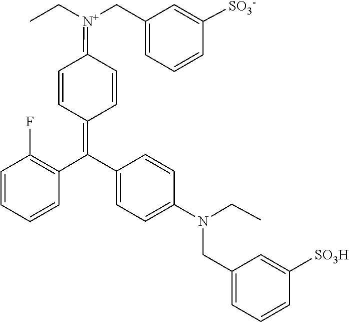 | [12] |
| 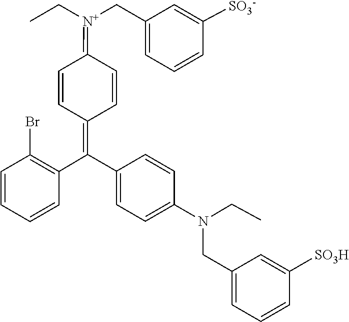 | [13] |
| 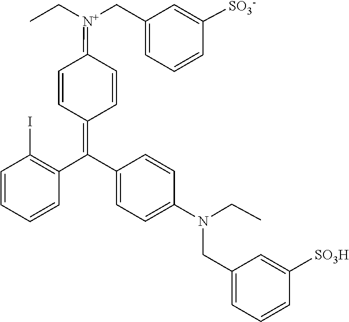 | [14] |
| 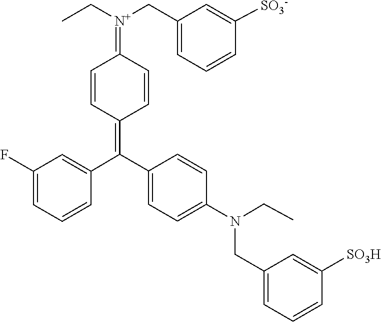 | [15] |
| 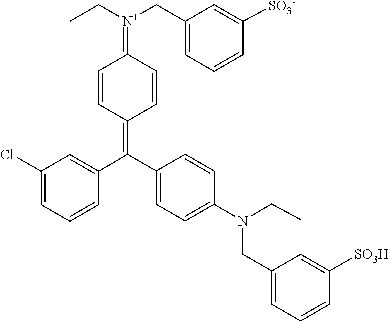 | [16] |
| 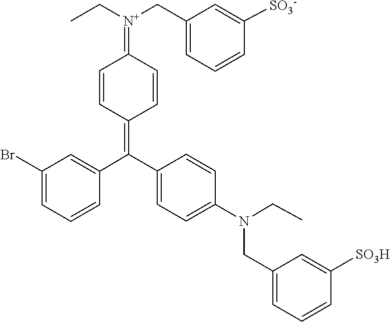 | [17] |
| 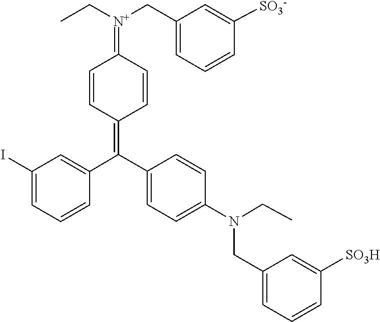 | [18] |
| 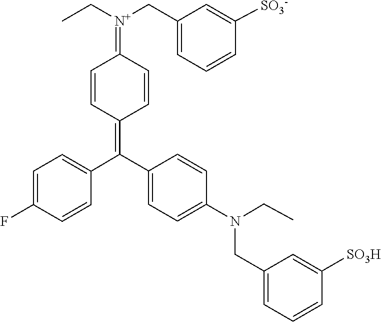 | [19] |
| 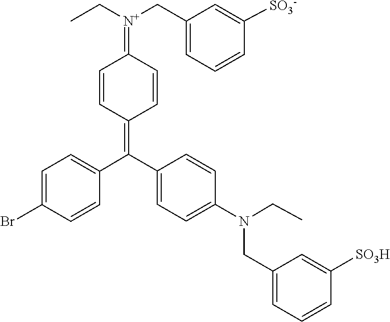 | [20] |
| 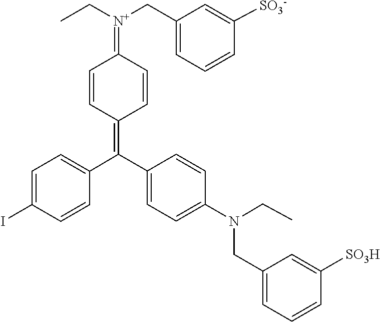 | [21] |

TABLE 3

| | |
|---|---|
| 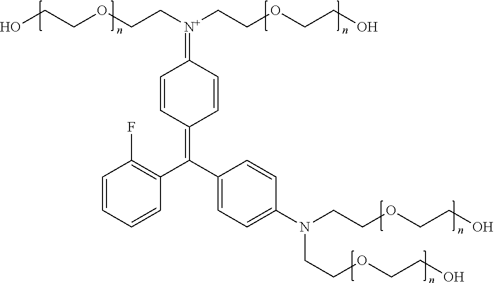 | [22] |
| 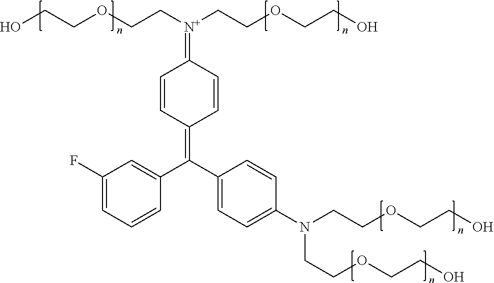 | [23] |
| 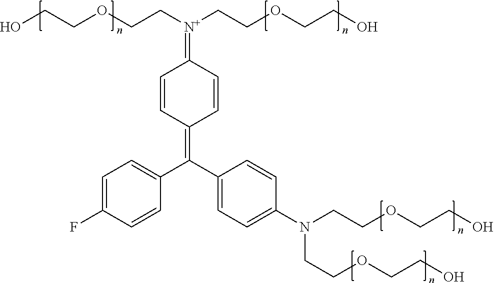 | [24] |
| 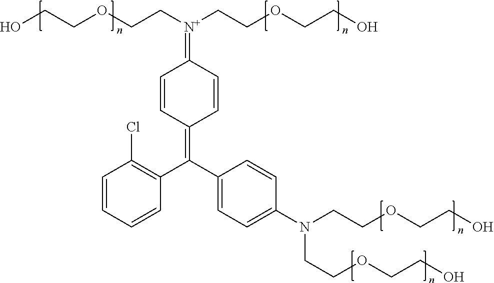 | [25] |
| 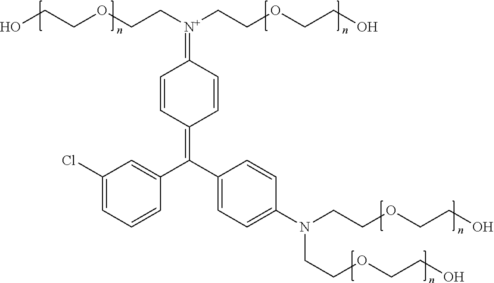 | [26] |
| 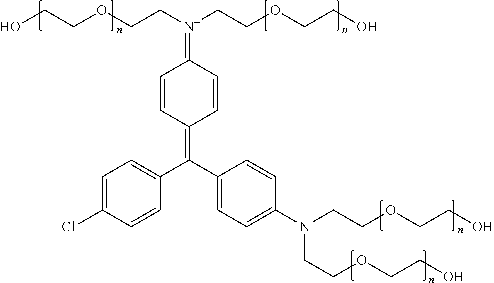 | [27] |
| 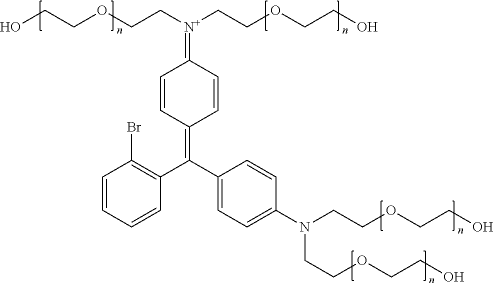 | [28] |
| 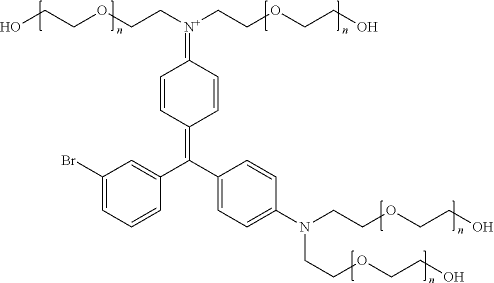 | [29] |
| 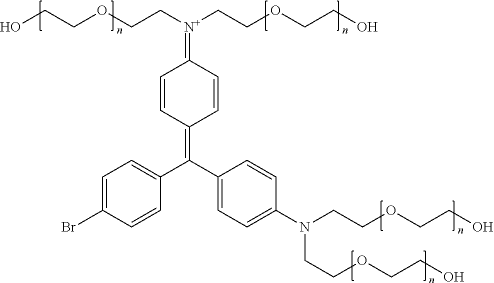 | [30] |
| 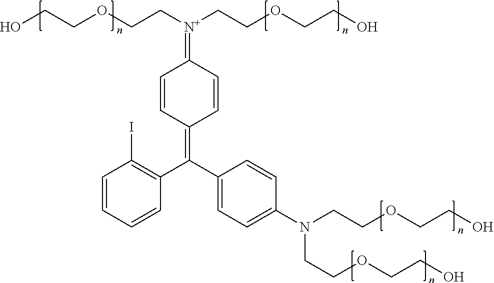 | [31] |
| 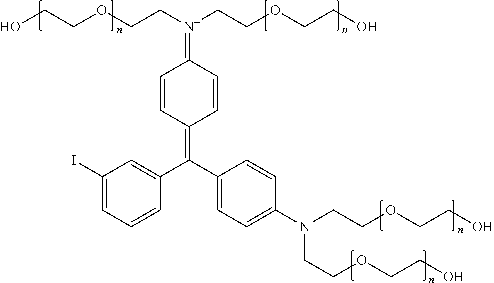 | [32] |
| 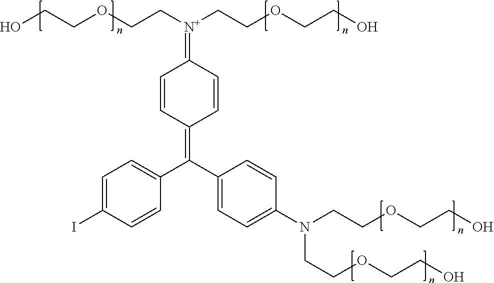 | [33] |

wherein n is 1-10, preferably 2-8.

TABLE 4

| | |
|---|---|
| 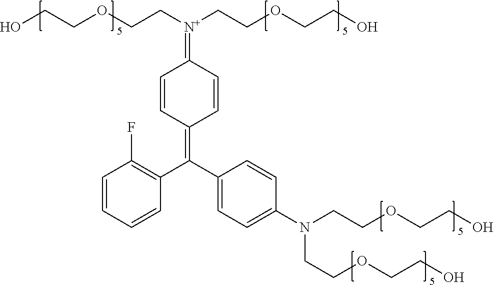 | [34] |
| 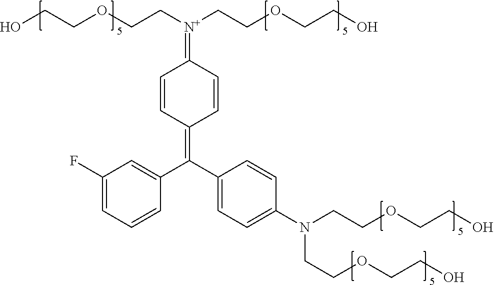 | [35] |
| 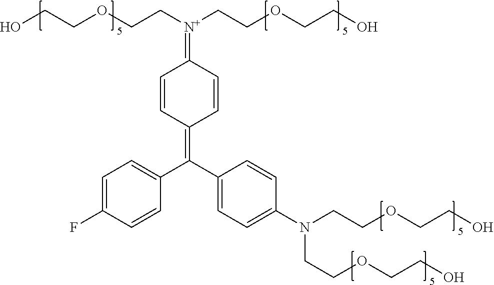 | [36] |
| 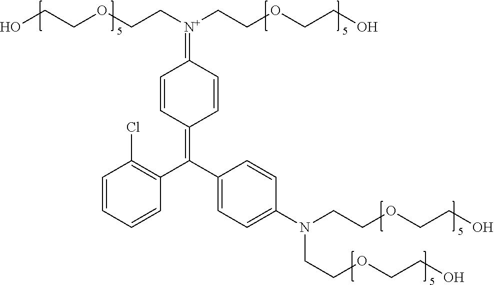 | [37] |
| 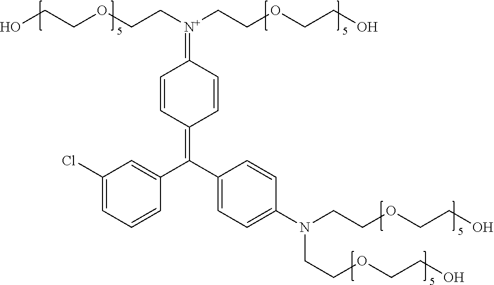 | [38] |
| 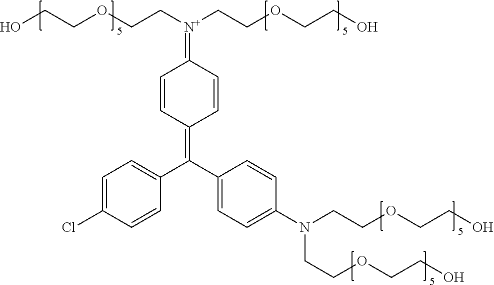 | [39] |
| 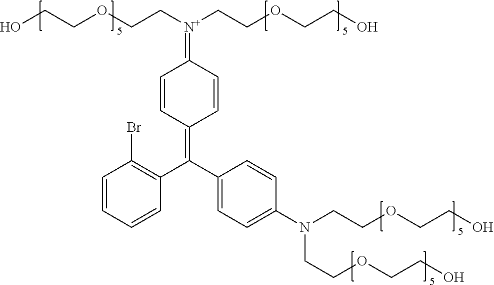 | [40] |
| 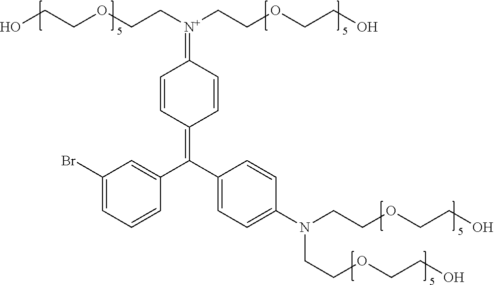 | [41] |
| 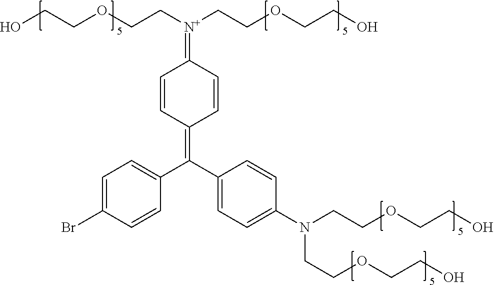 | [42] |
| 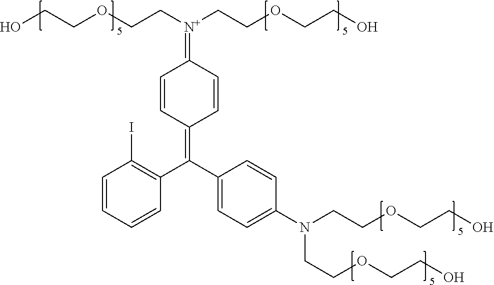 | [43] |
| 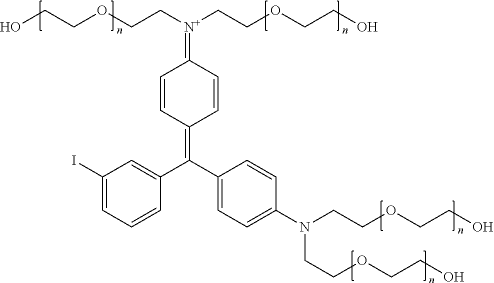 | [44] |
| 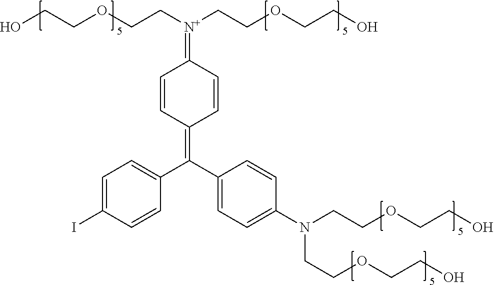 | [45] |

### Materials and Methods

### Materials

Acid Green 9 was purchased from TCI chemicals and used without further purification. N-Ethyl-N-benzyl aniline-3′-sulfonic acid was purchased from AK Scientific. Ethoxylated aniline was purchased from Ethox Chemicals. Halogenated aldehydes and p-toluene sulfonic acid (PTSA) were all purchased from Sigma Aldrich. N,N-dimethyl formamide (DMF), ethanol and chloranil were purchased from VWR. Deuterated solvents were purchased from Cambridge Isotope Laboratories Inc. Hydrogen peroxide stock solution was purchased from Evonik and diluted with DI water to 0.5% w/w before use.

### Synthesis of Meta Chloro Acid Triarylmethane

To a 3-neck round bottom flask fitted with a condenser, 2 eq of N-Ethyl-N-benzyl aniline-3′-sulfonic acid, 1 eq of PTSA and DMF were combined and heated in an oil bath until solids fully dissolved. 1 eq of halogenated aldehyde was added and the mixture stirred overnight to yield the leuco dye. 1 eq of chloranil was added to the crude mixture. After 2.5 hours the round bottom was removed from heat. Solvents were removed in vacuo and the resulting solids were redissolved in methanol and purified by chromatography to yield the pure dye species.

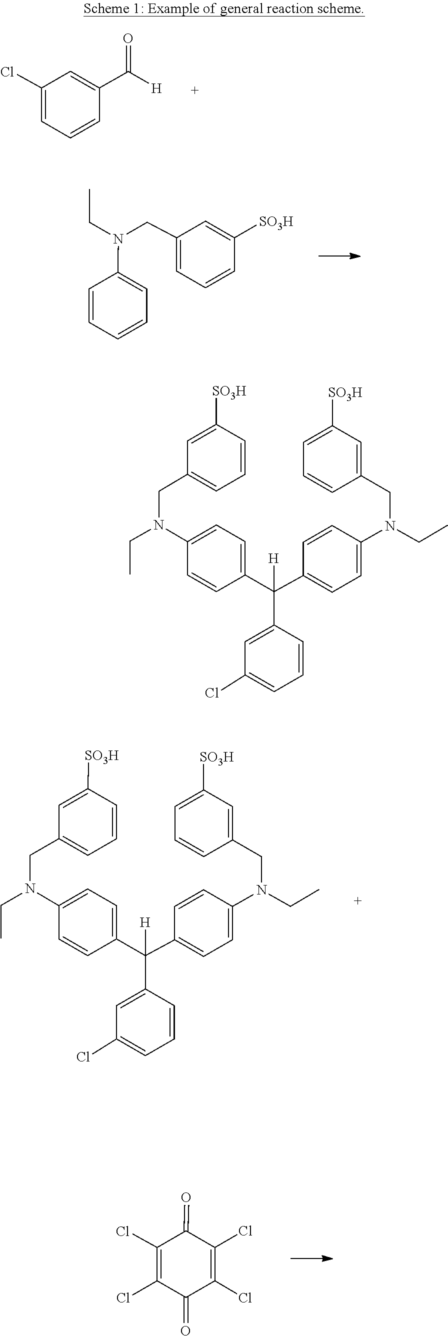

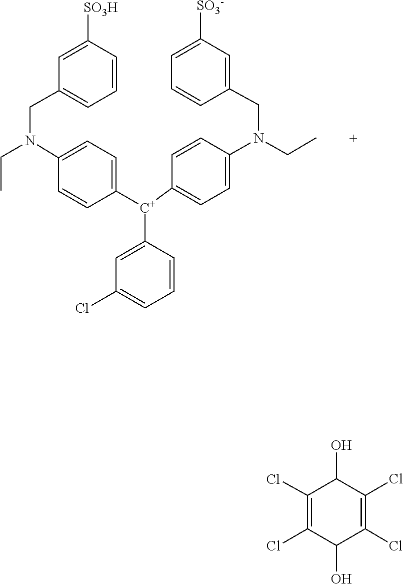

### Synthesis of Meta Chloro Polyethylene Glycol (PEG) Triarylmethane

In a beaker, 1 eq of ethanol was mixed with 3 eq of water, and the pH of the mixture was adjusted to 0.7 using concentrated HCl. To a 3-neck round bottom flask fitted with a condenser, 2 eq of ethoxylated aniline, 1 eq of halogenated aldehyde were dissolved in the acidic ethanol/water mixture. The mixture was heated and stirred to yield the leuco dye. 1.1 eq of chloranil was added to the crude mixture. After 18 hours the round bottom was removed from the heat. Solvents were removed in vacuo and the dye was redissolved in methanol and purified by chromatography to yield the pure dye species.

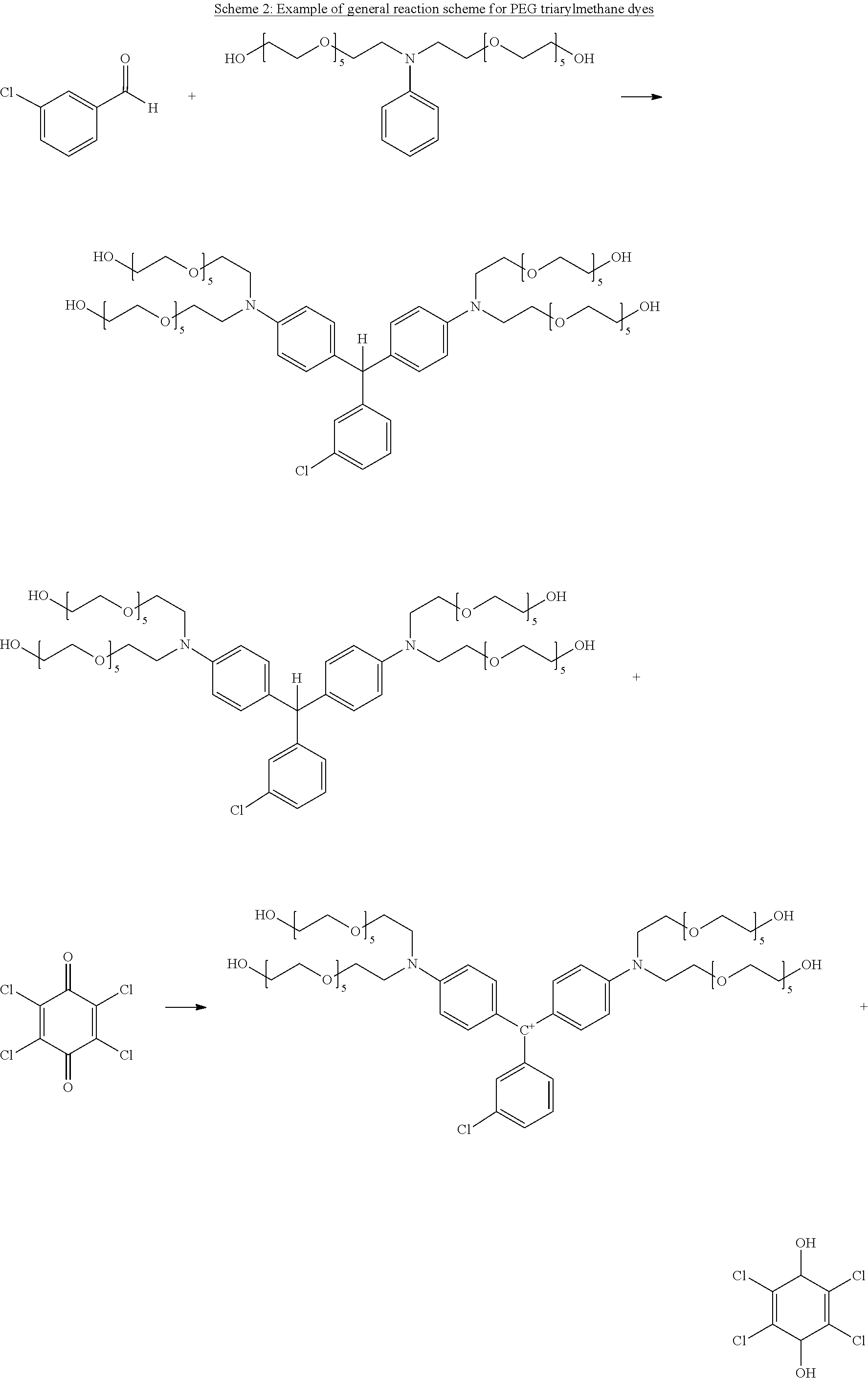

### Fade Tests

Light absorption measurements were taken with a Shimadzu UV-VIS 1900i spectrometer. Dyes were dissolved in unbuffered DI water to make up 10 mM stock solutions.

To measure dye fade rate in basic conditions, 25 μL of stock dye solution was added to 10 mL of 0.1M pH=8 potassium phosphate buffer. 3 mL of the pH=8 diluted dye solution were then pipetted into a clean quartz cuvette and inserted into spectrometer. Absorbance intensity at lambda max was measured every second for 400 s for each of the dyes studied.

To measure dye sensitivity to hydrogen peroxide, 25 μL of stock dye solution was added to 10 mL of DI water. 1.5 mL of dilute stock solution was added to a clean quartz cuvette. To the same cuvette added 1.5 mL of 0.5% w/w hydrogen peroxide solution. Cuvette was then briefly vortexed to ensure even mixing and loaded into spectrometer. Absorbance intensity at lambda max was measured every second for 400 s for each of the dyes studied.

### NMR Characterization

¹H NMR samples were dissolved in Methanol D-4 and scans were acquired using a 300 MHz NMR.

### Mass Spectrometry

Mass characterization data was acquired with a high-resolution mass spectrometer.

### Results

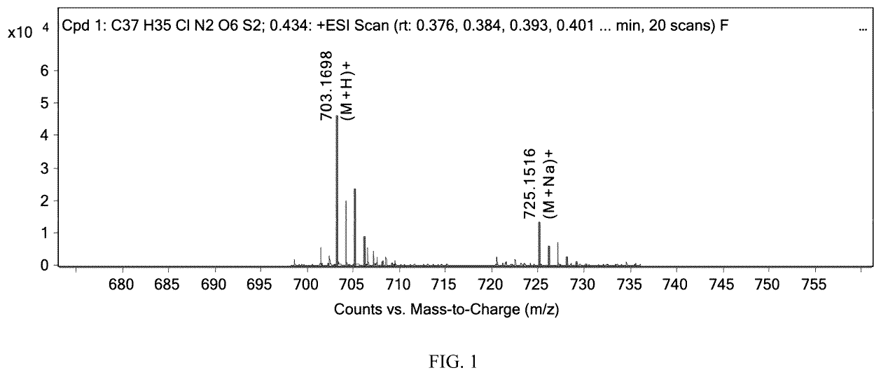

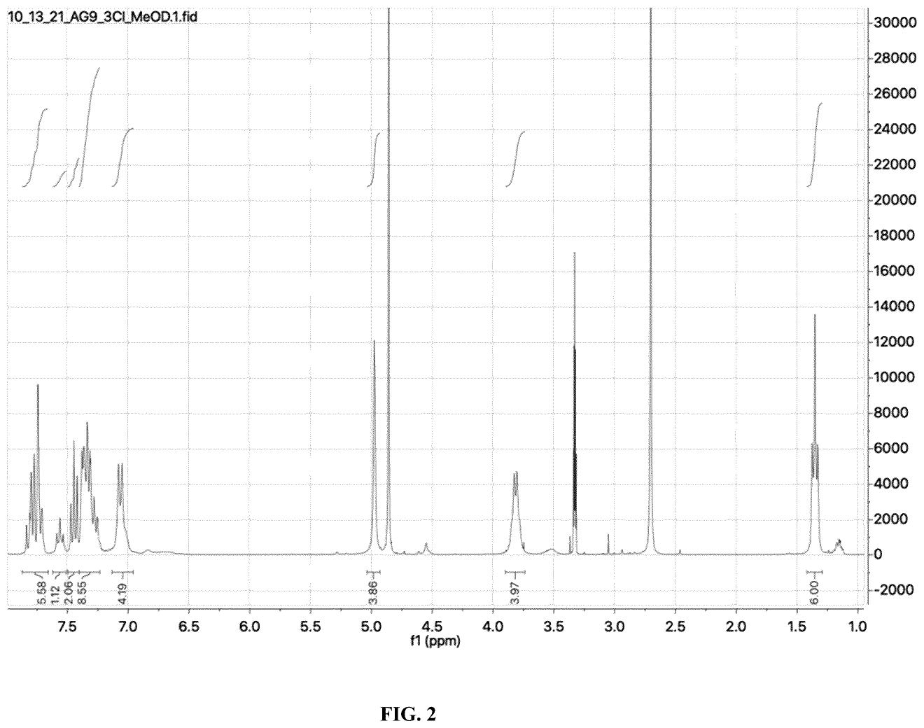

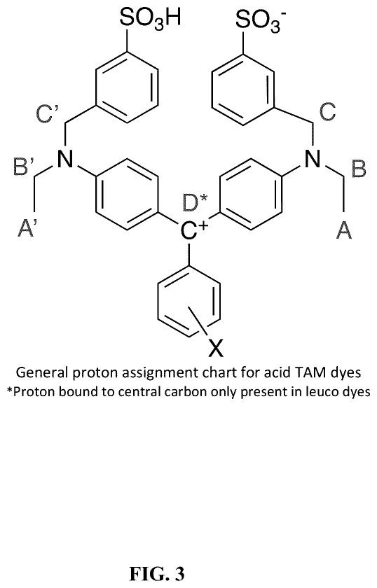

Meta chloro acid triarylmethane was shown to have the theorized mass and structure via ¹H NMR and high-resolution mass spectrometry (FIG. 1-FIG. 3 and Table 5 (below)).

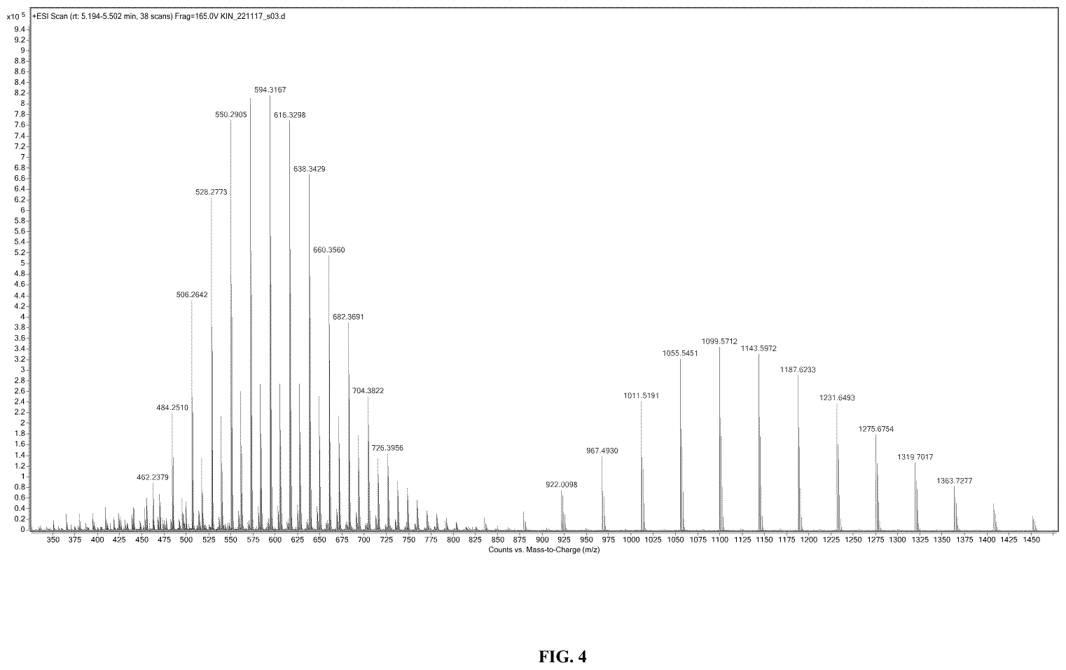

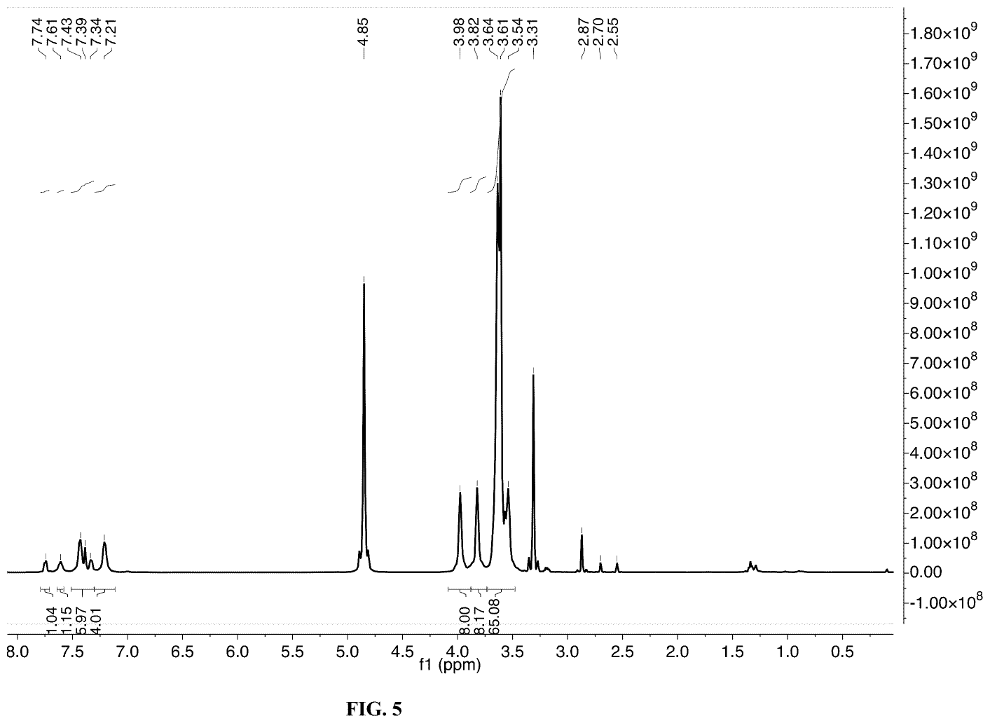

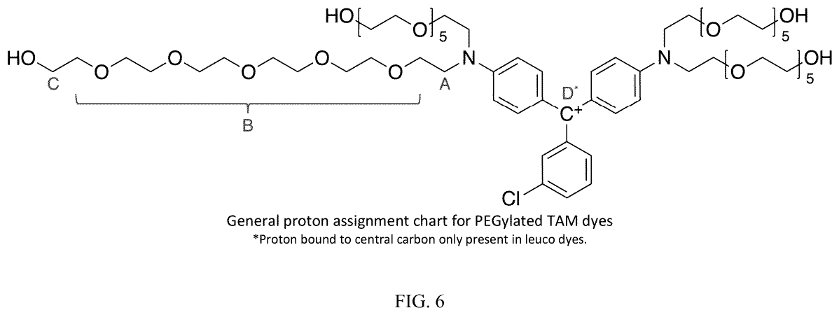

Meta chloro PEG triarylmethane was shown to have the theorized mass and structure via ¹H NMR and high-resolution mass spectrometry (FIG. 4-FIG. 6 and Table 6 (below)).

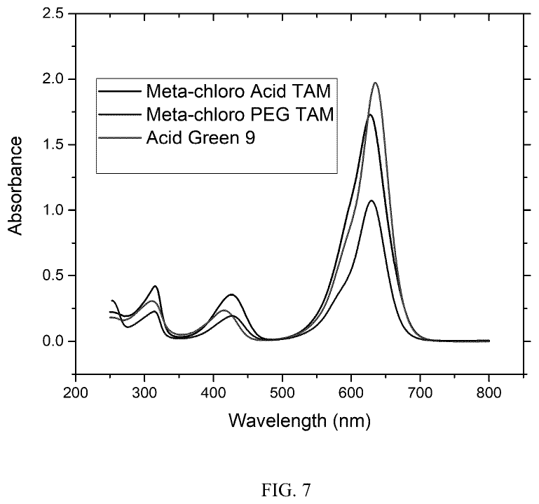

In solution, both meta chloro acid triarylmethane and meta chloro PEG triarylmethane were a greener shade of blue than Acid Green 9. FIG. 7 and Table 7 (below) depicts a clear shift in absorbance maximum and relative absorbance intensities between meta chloro acid triarylmethane, meta chloro PEG triarylmethane and Acid Green 9. Such a difference in the absorption spectrum would likely correspond to a change of perceived color in solution.

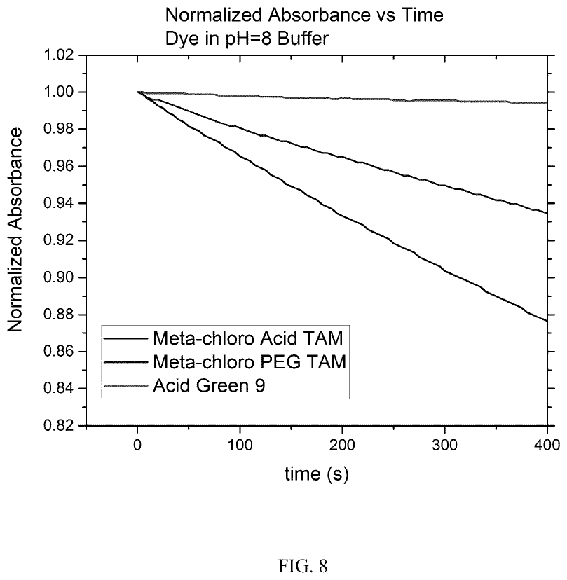

Meta chloro acid triarylmethane and meta chloro PEG triarylmethane both showed decreased stability in alkaline pH when compared to Acid Green 9. A pH=8, 25 μM solutions of both meta chloro acid triarylmethane and meta chloro PEG triarylmethane showed considerable fading in a 400 s time window, whereas Acid Green 9 in the same conditions showed negligible fading (FIG. 8 and Table 8 (below)).

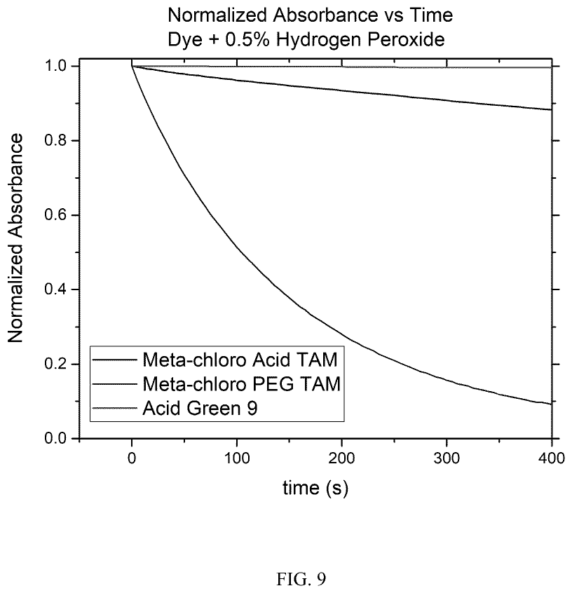

Meta chloro acid triarylmethane and meta chloro PEG triarylmethane both showed an increased rate of fading in hydrogen peroxide when compared to Acid Green 9. Unbuffered solutions of both meta chloro acid triarylmethane and meta chloro PEG triarylmethane showed considerable fading in a 400 s time window when combined with 0.5% w/w hydrogen peroxide. Acid Green 9 showed negligible fading in the same time window when combined with such low concentrations of hydrogen peroxide (FIG. 9 and Table 9 (below))

TABLE 5
NMR peak assignments for meta chloro acid triarylmethane dye

| | A/A′ shift | B/B′ shift | C/C′ shift | D* shift |
|---|---|---|---|---|
| Dye | (ppm) | (ppm) | (ppm) | (ppm) |
| Meta chloro acid | 1.35 | 3.80 | 4.98 | NA |
| triarylmethane | | | | |

TABLE 6
NMR peak assignments for meta chloro PEG triarylmethane dye

| | A shift | B shift | C shift | D shift |
|---|---|---|---|---|
| Dye | (ppm) | (ppm) | (ppm) | (ppm) |
| Meta chloro PEG | 3.98 | 3.61 | 3.82 | NA |
| triarylmethane | | | | |

TABLE 7
Lambda max position and absorbance values for meta
chloro acid triarylmethane dye and Acid Green 9

| Dye | λmax(nm) | Absorbance |
|---|---|---|
| Acid Green 9 | 635 | 1.969 |
| Meta chloro acid triarylmethane | 628 | 1.728 |
| Meta chloro PEG triarylmethane | 630 | 1.074 |

TABLE 8
Fade rate at lambda max of 25 μM dye solution in pH = 8 buffer

| | Fade Rate in pH 8 |
|---|---|
| Dye | buffer (mAbs/min) |
| Acid Green 9 | −0.001 |
| Meta chloro Acid triarylmethane | −0.014 |
| Meta chloro PEG triarylmethane | −0.048 |

TABLE 9
Fade rate of 25 μM dye combined with 0.5% hydrogen peroxide

| | Fade Rate w/ |
|---|---|
| Dye | 0.5% (w/w) H₂O₂ |
| Acid Green 9 | 0.000 |
| Meta chloro acid triarylmethane | −0.013 |
| Meta chloro PEG triarylmethane | −0.663 |

While this invention has been particularly shown and described with references to preferred embodiments thereof, it will be understood by those skilled in the art that various changes in form and details may be made therein without departing from the scope of the invention encompassed by the appended claims.

1-11. (canceled)

12. A modified triarylmethane dye comprising the structure shown in Formula 4, 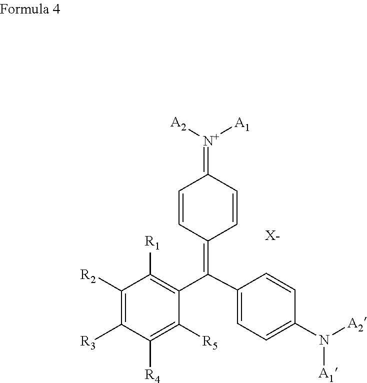
wherein
R₁, R₂, R₃, R₄, and R₅ are independently selected from hydrogen, halogen, C₁-C₁₆ alkyl, amino, nitro, methoxy, carboxy, aryl, alkoxy, or sulfonic acid, provided that at least one of R₁, R₂, or R₃ are halogen; and
A₁, and A_{1′} are alkyl or alkoxy; and A₂, and A_{2′} are alkyl, alkaryl, or alkoxy; wherein the alkyl, alkaryl, or alkoxy are optionally substituted.

13. The modified triarylmethane dye according to claim 12, wherein R₁, R₂, R₃, R₄, and R₅ are independently selected from hydrogen or halogen, provided that at least one of R₁, R₂, or R₃ are halogen.

14. The modified triarylmethane dye according to claim 12, wherein (i) A₁ and A₁′ are alkyl and A₂ and A₂′ are alkyl substituted with aryl; or (ii) wherein A₁ and A₁′ are alkyl substituted with alkoxy and A₂ and A₂′ are alkyl substituted with alkoxy, wherein the aryl and the alkoxy are optionally substituted.

15. The modified triarylmethane dye according to claim 12, wherein only one of R₁, R₂, or R₃ is halogen, and wherein:
(i) when R₁ is halogen, R₂ and R₃ are hydrogen and R₁ is selected from fluorine, bromine, chlorine, or iodine,
(ii) when R₂ is halogen, R₁ and R₃ are hydrogen and R₂ is selected from fluorine, bromine, chlorine, or iodine, or
(iii) when R₃ is halogen, R₁ and R₂ are hydrogen and R₃ is selected from fluorine, bromine, chlorine, or iodine.

16. The modified triarylmethane dye according to claim 15, wherein R₁ is halogen.

17. The modified triarylmethane dye according to claim 15, wherein R₂ is halogen.

18. The modified triarylmethane dye according to claim 15, wherein R₃ is halogen.

19. The modified triarylmethane dye according to claim 12, wherein A₁ and A₁′ are the same and A₂ and A₂′ are the same.

20. The modified triarylmethane dye according to claim 14, wherein A₁ and A₁′ are alkyl and A₂ and A₂′ are alkyl substituted with aryl.

21. The modified triarylmethane dye according to claim 20, wherein A₁ and A₁′ are a C₁-C₈alkyl and A₂ and A₂′ are a C₆-C₁₂ aryl attached to N via a bridging alkyl carbon.

22. The modified triarylmethane dye according to claim 21, wherein A₁ and A₁′ are ethyl and the C₆-C₁₂ aryl of A₂ and A₂′ is benzyl sulfonic acid.

23. The modified triarylmethane dye according to claim 19, wherein R₂ is halogen.

24. The modified triarylmethane dye according to claim 23, wherein R₂ is chlorine.

25. The modified triarylmethane dye according to claim 12, wherein A₁, A₁′, A₂, and A₂′ are alkyl substituted with alkoxy.

26. The modified triarylmethane dye according to claim 25, wherein the alkoxy of A₁, A₁′, A₂, and A₂′ are polyethylene glycol.

27. The modified triarylmethane dye according to claim 26, wherein A₁, A₁′, A₂, and A₂′ are independently polyethylene glycol with 1-10 ethylene glycol repeat units.

28. The modified triarylmethane dye according to claim 27, wherein A₁, A₁′, A₂, and A₂ are polyethylene glycol with 5 ethylene glycol repeat units.

29. The modified triarylmethane dye according to claim 25, wherein R₂ is halogen.

30. The modified triarylmethane dye according to claim 29, wherein R₂ is chlorine.

31. The modified triarylmethane dye according to claim 29, wherein R₁ and R₃ are hydrogen.

32-41. (canceled)
